(19) United States
(12) Patent Application Publication
(10) Pub. No.: US 2023/0190598 A1
(43) Pub. Date: Jun. 22, 2023

(54) HAIRSPRAY FORMULATIONS FOR HFO PROPELLANTS

(71) Applicant: Honeywell International Inc., Charlotte, NC (US)

(72) Inventors: Gregory L. Smith, Ontario (CA); Anthony M. Anzalone, Angola, NY (US); Barry Setiawan, East Amherst, NY (US); Ryan J. Hulse, Getzville, NY (US)

(21) Appl. No.: 18/080,312

(22) Filed: Dec. 13, 2022

Related U.S. Application Data
(60) Provisional application No. 63/290,132, filed on Dec. 16, 2021.

(51) Int. Cl. A61K 8/31; A61K 8/34; A61K 8/41; A61K 8/85; A61K 8/04; A61Q 5/06

(52) U.S. Cl. CPC A61K 8/315; A61K 8/34; A61K 8/41; A61K 8/046; A61K 8/85; A61Q 5/06

(57) ABSTRACT

The present disclosure provides a formulation comprising a fluoroolefin, an amine neutralizer, a fixative polymer, and a solvent. The formulation may be trans-1,3,3,3-tetrafluoropropene (HFO-1234ze(E)), and the amine neutralizer may be triethanolamine (TEA), triisopropanolamine (TIPA), or a mixture thereof. The formulation may be suitable for use as a personal care product, such as a hair spray.

### CROSS-REFERENCE TO RELATED APPLICATION

This application claims priority to U.S. Provisional Application No. 63/290,132, filed Dec. 16, 2021, which is herein incorporated by reference in its entirety.

### FIELD

The present invention relates to cosmetic formulations, including particularly hair treatment formulations and methods of making such formulations that are environmentally friendly and stable.

### BACKGROUND

Hair treatment formulations, and in particular hair sprays, are now widely used, and are usually formulations which contain one or more pre-formed polymeric materials (sometimes referred to as resins) which are included for the purpose of imparting a desired characteristic to the hair. For example, polymers may be used to give hair shine, style hair, preserve hair style, give hair a desired texture or feel, enhance hair color, condition hair, dry hair so it sets quickly, straighten or smooth hair, soften hair, strengthen hair, make hard-to-treat hair manageable, enhance optical properties, provide hold to hair, provide frizz control and/or to repair damaged hair. The polymers may be fixative components which serve to provide “hold” to the hair, that is, to help maintain the hair in the desired style or shape. Such polymers are usually included in the formulation with one or more other components, including a propellant.

The propellant in such hair treatment formulations enables the polymer component to be applied to the hair in a finely dispersed spray, and once applied to the hair the polymer component performs its intended function in connection with the hair. For example, a hair spray that includes a fixative will tend to add some stiffness to the hair so that it holds the desired shape.

It has heretofore been common practice to use propellants that have one or more detrimental impacts when released into the environment. For example, many of the previously and currently used propellants have high global warming potential (GWP), and are volatile organic compounds (VOCs). It has been widely accepted that compounds with these properties have an adverse effect on the earth’s atmosphere and/or environment. As a result, there has been a drive both commercially and governmentally to reduce the use of such materials in many applications, including as propellants.

While several compounds and blends of compounds have been suggested for use as propellants in various applications, selecting propellants for use in hair treatment formulations is especially complex as the substitution of a new propellant into a formulation in order to improve environmental performance can result in a deterioration in one or more other properties of the hair treatment formulation. For example, it has been found that certain types of hair treatment polymers and formulations can degrade or deteriorate in performance when exposed to certain environmentally advantageous propellants.

Improvements to the foregoing are desired.

### SUMMARY

The present disclosure provides a formulation comprising a fluoroolefin of the formula

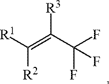

wherein R¹ is hydrogen or fluorine, R² is hydrogen or fluorine, R³ is hydrogen or fluorine; a solvent; an amine neutralizer; and a fixative polymer. The fluoroolefin may have a global warming potential (GWP), as measured in carbon dioxide (CO₂) equivalents, of 10 or less, such as 10 or less, 9 or less, 8 or less, 6 or less, 5 or less, 4 or less, 3 or less, 2 or less, or 1 or less. Suitable fluoroolefins may include trans-1,3,3,3-tetrafluoropropene (HFO-1234ze(E)).

The present disclosure further provides personal care products, such as hairspray, comprising the formulations provided herein. The present disclosure also provides methods of making the formulations provided herein.

### DETAILED DESCRIPTION

### 1. Definitions

The singular forms “a”, “an”, and “the” include plural forms unless the context clearly dictates otherwise specified or clearly implied to the contrary by the context in which the reference is made.

The term “about” as used herein generally indicate a relative difference of 10 percent of the value specified. Numerical ranges as used herein are meant to include every number and subset of numbers enclosed within that range, whether particularly disclosed or not. Further, these numerical ranges are intended to be understood as providing support for a claim directed to any number or subset of numbers in that range.

All percentages, parts, proportions and ratios as used herein, are by weight of the total of the indicated formulation, unless otherwise specified.

All references to singular characteristics or limitations of the present invention shall include the corresponding plural characteristic or limitation, and vice-versa, unless otherwise specified or clearly implied to the contrary by the context in which the reference is made.

As used herein, the words “preferred” or “preferably” and variants refer to embodiments of the invention that afford certain benefits, under certain circumstances. However, other embodiments may also be preferred, under the same or other circumstances. Furthermore, the recitation of one or more preferred embodiments does not imply that other embodiments are not useful, and is not intended to exclude other embodiments from the scope of the invention.

As used herein, the term “homopolymer” is intended to include those polymers which are formed essentially only from a single monomeric structures.

As used herein, the term “copolymer” is intended to include those polymers which are formed from two or more monomeric structures, and thus include those polymers which are formed from three monomeric structures (also known as terpolymers) and so on.

### 2. Hairspray Formulations

The formulations of the present disclosure may be suitable for use in personal care products, such as hairspray. These formulations may include a one or more propellants, one or more fixative polymers, one or more solvents, and one or more amine neutralizers. The amine neutralizer may improve the performance of the fixative polymer. However, the amine neutralizer may be capable of reacting with the propellant, particularly in cases wherein the propellant comprises an olefin. Thus, the selection of an appropriate combination of amine neutralizer and propellant may be challenging as many combinations may be chemically unstable. The present disclosure provides a stable formulation in which the amine neutralizer does not react significantly with the olefin, while maintaining the performance of the fixative polymer. Each component of the formulation is discussed in further detail below.

### 3. Propellant

Aerosol propellants often comprise fluorocarbon-based compounds. The industry is continually seeking new fluorocarbon-based mixtures that offer alternatives, and are considered environmentally safer substitutes for CFCs, HCFCs and HFCs in use today. Of particular interest are mixtures containing hydrofluorocarbons, fluoroolefins, iodide-containing compounds and other fluorinated compounds, which have low ozone depletion potentials and low global warming potentials.

The present disclosure provides formulations which include propellants with lower global warming potential (GWP) than those currently used in aerosolized formulations, such as 1,2-difluoroethane (HFC-152a) for example. GWP was developed to allow comparisons of the global warming impact of different gases. Specifically, it is a measure of how much energy the emission of one ton of a gas will absorb over a given period of time, relative to the emission of one ton of carbon dioxide. The larger the GWP, the more that a given gas warms the Earth compared to CO₂ over the same time period. The time period used for GWP is 100 years. GWP provides a common measure, which allows analysts to add up emission estimates of different gases. 1,2-Difluoroethane (HFC-152a), for example, has a GWP of 138.

By way of comparison, the fluoroolefins included in the formulations of the present disclosure have a global warming potential (GWP), as measured in carbon dioxide equivalents, of 10 or less, 9 or less, 8 or less, 7 or less, 6 or less, 5 or less, 4 or less, 3 or less, 2 or less, or 1 or less.

The formulations of the present disclosure may include fluoroolefins of Formula I, shown below:

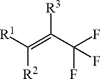

wherein R¹ is hydrogen or fluorine, R² is hydrogen or fluorine, and R³ is hydrogen or fluorine.

Specific fluoroolefins for use in the formulations of the present disclosure may include trans-1,3,3,3-tetrafluoropropene (HFO-1234ze(E)).

The fluoroolefin may be present in the formulation in an amount of about 10 wt.% or greater, about 15 wt.% or greater, about 20 wt.% or greater, about 25 wt.% or greater, 30 wt.% or greater, about 35 wt.% or greater, about 40 wt.% or greater, about 45 wt.% or less, about 50 wt.% or less, about 55 wt.% or less, about 60 wt.% or less, about 65 wt.% or less, about 70 wt.% or less, about 75 wt.% or less, about 80 wt.% or less, about 85 wt.% or less, about 90 wt.% or less, or any value encompassed by these endpoints, based on the total weight of the formulation.

The formulations of the present disclosure may further comprise co-propellants. Suitable co-propellants may include 1,1-difluoroethane (HFC-152a); dimethyl ether; hydrocarbons such as propane, isobutane, and n-butane; and compressed gases such as nitrogen, air, and carbon dioxide, for example.

### 4. Fixative Polymer

The formulations of the present disclosure may vary over a wide range according to the particular needs or target properties for the formulation. For example, the formulation may be used in a personal care formulation, such as a hair spray in which the fixative polymer may be present in an amount of about 4 wt.% to 8 wt.%, or a texturizing spray in which the fixative polymer may be present in an amount of about 1 wt.% to 2 wt.%. Accordingly, the formulations of the present disclosure may include fixative polymers suitable for hair treatment.

Suitable fixative polymers for use in the formulations of the present disclosure may include copolymers. The copolymers may comprise two-component copolymers, such as acrylate-octylacrylamide copolymers, for example. The copolymers may comprise three-component copolymers, such as vinyl acetate-crotonate-vinyl neodecanoate co-polymers, for example. The copolymers may comprise four-component copolymers, such as octylacrylamide-acrylate-butylaminoethyl-methacrylate copolymer, for example.

The fixative polymer may be described by its acid value. The acid value of the polymer is defined as the mass of potassium hydroxide (KOH) in milligrams needed to neutralize one gram of the polymer, as described in ASTM D 974, for example. Suitable fixative polymers for the formulations of the present disclosure have an acid value of 2.05 meq/g.

The fixative polymer may be present in the formulation in an amount of 1 wt.% or greater, about 2 wt.% or greater, about 3 wt.% or greater, about 5 wt.% or greater, about 7 wt.% or greater, about 10 wt.% or less, about 12 wt.% or less, about 15 wt.% or less, about 17 wt.% or less, about 20 wt.% or less, or any value encompassed by these endpoints, based on the total weight of the formulation.

### 5. Amine Neutralizer

As described above, many hairspray formulations use an amine neutralizer to impart performance attributes to the polymer on the hair. However, amines may react easily with olefins, such as the low GWP propellant trans-1,3,3,3-tetrafluoropropene (HFO-1234ze(E)), used in the formulations of the present disclosure.

One widely used neutralizer is aminomethyl propanol (AMP); a primary amine that further imparts good performance characteristics to the fixative polymer. However, as described further below, AMP was found to react with the low GWP propellant trans-1,3,3,3-tetrafluoropropene (HFO-1234ze(E)), as did monoisopropanolamine (MIPA), another widely used amine neutralizer.

The reactivity of amines with olefins may be more likely when primary amines are used and due to lack of steric hinderance at the nitrogen atom. In contrast, sterically hindered tertiary amines are expected to be less reactive with olefins. However, when tertiary amines are used in formulations for hairspray, the performance of the fixative polymer on the hair is often unacceptable. Without wishing to be bound by theory, it is thought that tertiary amines may have an adverse effect on the performance of fixative polymers.

It was surprisingly found that two tertiary amines – triethanolamine (TEA) and triisopropanolamine (TIPA) - performed well both with regard to their stability towards trans-1,3,3,3-tetrafluoropropene and their ability to impart good performance on fixative polymers.

The formulations of the present disclosure may include amine neutralizers in an amount of about 0.1 wt.% or greater, about 0.2 wt.% or greater, about 0.5 wt.% or greater, about 1.0 wt.% or greater, about 1.2 wt.% or less, about 1.5 wt.% or less, about 2.0 wt.% or less, or any value encompassed by these endpoints, based on the total weight of the formulation.

The stability of the mixture was tested by accelerated aging testing. The formulation was stored in a sealed container at 50° C. for a period of three months. Following the three-month aging period, the formulation was tested to determine the amount of fluoride present using an ion-selective electrode. A formulation was deemed stable if the amount of fluoride present was less than 300 ppm. For example, stable formulations included fluoride in an amount of 300 ppm or less, 250 ppm or less, 225 ppm or less, 200 ppm or less, 150 ppm or less, 100 ppm or less, 50 ppm or less, 10 ppm or less, or 5 ppm or less.

Following the three-month waiting period, the formulation was also observed to determine whether precipitates were present. A formulation was deemed stable if there were no precipitates present upon visual inspection.

### 6. Solvent

The formulations of the present disclosure may include one or more solvent. Suitable solvents may include water and low molecular weight alcohols, such as ethanol and isopropanol, for example.

The solvent may be present in the formulation in an amount of about 10 wt.% or greater, about 15 wt.% or greater, about 20 wt.% or greater, about 25 wt.% or greater, about 30 wt.% or greater, about 35 wt.% or greater, about 40 wt.% or greater, about 45 wt.% or greater, about 50 wt.% or less, about 55 wt.% or less, about 60 wt.% or less, about 65 wt.% or less, about 70 wt.% or less, about 75 wt.% or less, about 80 wt.% or less, about 85 wt.% or less, about 90 wt.% or less, or any value encompassed by these endpoints, based on the total weight of the formulation.

Suitable co-solvents for use in the formulations of the present disclosure may include water, ethanol, isopropanol, and mixtures thereof, for example.

The co-solvent may be present in the formulation in an amount of 0 wt.% or greater, about 1 wt.% or greater, about 5 wt.% or greater, about 10 wt.% or greater, about 20 wt.% or greater, about 30 wt.% or greater, about 40 wt.% or greater, about 50 wt.% or greater, about 60 wt.% or greater, about 70 wt.% or greater, about 80 wt.% or greater, about 90 wt.% or greater, about 95 wt.% or greater, about 99 wt.% or greater, or any value encompassed by these endpoints, based on the total weight of the formulation.

### 7. Additives

The formulations of the present disclosure may further comprise additives. Additives may include one or more of each of lubricants, stabilizers, corrosion inhibitors, fragrances, starches, and performance modifiers. Suitable performance modifiers may include silicones, for example.

### 8. Method of Making

The formulations of the present disclosure may be synthesized by first adding the fixative polymer slowly to the solvent at room temperature with stirring. Other components, such as the amine neutralizer and desired additives, are then added while continuing to stir the mixture. Finally, the propellant is added to complete the formulation.

### 9. Method of Use

The formulation of the present disclosure may be suitable for use as personal care products. In one aspect, the personal care product may be a hair treatment product. When used in this manner, the formulation may be applied to hair in order to impart desired characteristics, such as style-ability, volume, shine, hair manageability, durability of style, softness, ease of styling, anti-frizz, hair fiber alignment, greater wet conditioning, dry conditioning, wet combing, ease of use with styling appliances, or hair protection from styling appliances. In one aspect, the formulations of the present disclosure may be used as a hair spray. Efficacy of these formulations may be determined by evaluating curl retention, stiffness, combability, feel, and washability.

The following non-limiting example serves to illustrate the disclosure.

### EXAMPLES

### Example 1: Reactivity of Amines Towards 1,1-difluoroethane (HFC-152a) and Trans-1,3,3,3-tetrafluoropropene (HFO-1234ze(E))

Four different amine neutralizers (two primary and two tertiary) were formulated with an octylacrylamide/acrylates/butylaminoethyl methacrylate copolymer in an amount of 5 wt.%, a solvent in an amount of 50 wt.%, and 45-x wt.% 1234ze with x wt.% amine neutralizer (where x = approx. 1). The formulations were then tested for stability alongside formulations including 1,1-difluoroethane (HFC-152a) – the current industry standard - instead of trans-1,3,3,3-tetrafluoropropene (HFO-1234ze(E)).

As shown below in Table 1, stability and performance may vary significantly from one propellant to another, and from one amine to another.

TABLE 1

| Neutralizer | With 152a | | With 1234ze(E) | |
|---|---|---|---|---|
| | Stability | Performance | Stability | Performance |
| 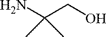 | Acceptable | Acceptable | Unacceptable | Not tested |
| Aminomethyl propanol (AMP) | | | | |
| 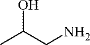 | Acceptable | Not tested | Unacceptable | Not tested |
| 1-Amino 2-propanol (MIPA) | | | | |
| 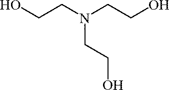 | Acceptable | Questionable | Acceptable | Acceptable |
| Triethanolamine (TEA) | | | | |
| 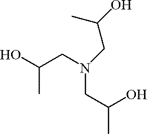 | Acceptable | Questionable | Acceptable | Acceptable |
| Triisopropanolamine (TIPA) | | | | |

### Example 2: Reactivity of Amines Towards Trans-1,3,3,3-tetrafluoropropene (HFO-1234ze(E))

Eight different neutralizers were tested for their stability towards trans-1,3,3,3-tetrafluoropropene (HFO-1234ze(E)) by detection of fluoride via an ion-selective electrode and visual observation of precipitate formation, as well as their performance on hair if the stability results were favorable. The results are shown below in Table 2.

TABLE 2

| | | |
|---|---|---|
| Neutralizer | Stability with 1234ze(E) | Performance on hair |
| KOH | Unacceptable | Not tested |
| 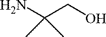 Aminomethyl propanol (AMP) | Unacceptable | Not tested |
| 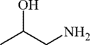 | Unacceptable | Not tested |
| 1-Amino 2-propanol (MIPA) | | |
| Aminomethyl propanediol (AMPD) | Unacceptable | Not tested |
| Triethanolamine (TEA) | Acceptable | Acceptable |
| Triisopropanolamine (TIPA) | Acceptable | Acceptable |
| 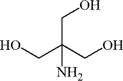 Tromethamine | Borderline | Acceptable |
| 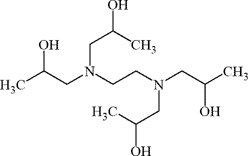 [SMILES: CC(O)CN(CCN(CC(C)O)CC(C)O)CC(C)O] Tetrahydroxypropyl-ethylenediamine (Neutrol TE) | Borderline | Acceptable |

In the stability testing of Tromethamine and Neutrol TE, it was found that while no precipitates were formed, the fluoride levels were at higher levels which in our experience are usually accompanied by precipitate formation, therefore, the stability was deemed borderline. Both of these cases are notable in that previous to testing Tromethamine, a primary amine, all primary amines tested had clearly unacceptable stability, and conversely, previous to testing Neutrol TE, a tertiary amine, all tertiary amines had clearly acceptable stability, whereas both of these examples have borderline stability.

### ASPECTS

Aspect 1 is a formulation comprising a fluoroolefin of the formula

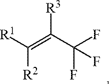

wherein R¹ is hydrogen or fluorine, R² is hydrogen or fluorine, R³ is hydrogen or fluorine; a solvent; an amine neutralizer; and a fixative polymer.

Aspect 2 is the formulation of Aspect 1, wherein the fluoroolefin has a GWP, as measured in carbon dioxide (CO₂) equivalents, of 5 or less.

Aspect 3 is the formulation of either Aspect 1 or Aspect 2, wherein the fluoroolefin comprises trans-1,3,3,3-tetrafluoropropene (HFO-1234ze(E)).

Aspect 4 is the formulation of any one of Aspects 1-3, wherein the solvent comprises ethanol.

Aspect 5 is the formulation of any one of Aspects 1-4, wherein the amine neutralizer comprises an amine that is unreactive towards HFO-1234ze(E) as determined by detection of fluoride via an ion-selective electrode.

Aspect 6 is the formulation of any one of Aspects 1-5, wherein the amine neutralizer comprises a tertiary amine.

Aspect 7 is the formulation of any one of Aspects 1-6, wherein the amine neutralizer comprises triethanolamine (TEA), triisopropanolamine (TIPA), or a mixture thereof.

Aspect 8 is the formulation of any one of Aspects 1-7, further comprising at least one of one or more co-propellants and one or more co-solvents.

Aspect 9 is the formulation of any one of Aspects 1-8, further comprising one or more additives selected from the group consisting of lubricants, stabilizers, corrosion inhibitors, fragrances, starches, and performance modifiers.

Aspect 10 is the formulation of any one of Aspects 1-9, wherein the fixative polymer is selected from the group consisting of acrylate-octylacrylamide copolymers, vinyl acetate-crotonate-vinyl neodecanoate co-polymers, and octylacrylamide-acrylate-butylaminoethyl-methacrylate copolymers.

Aspect 11 is the formulation of any one of Aspects 1-10, wherein the fluoroolefin is present in the formulation in an amount of 35 wt.% to 45 wt.% based on the total weight of the formulation.

Aspect 12 is the formulation of any one of Aspects 1-11, wherein the solvent is present in an amount of 45 wt.% to 55 wt.% based on the total weight of the formulation.

Aspect 13 is the formulation of any one of Aspects 1-12, wherein the amine neutralizer is present in the formulation in an amount of 0.2 wt.% to 1.5 wt.% based on the total weight of the formulation.

Aspect 14 is the formulation of any one of Aspects 1-13, wherein the fixative polymer is present in the formulation in an amount of 1 wt.% to 15 wt.% based on the total weight of the formulation.

Aspect 15 is a personal care formulation comprising the formulation of any one of Aspects 1-14.

Aspect 16 is the personal care formulation of Aspect 15, wherein the formulation is an aerosol.

Aspect 17 is the personal care formulation of either Aspect 15 or Aspect 16, wherein the formulation is a hairspray.

Aspect 18 is a method, comprising applying the formulation of Aspect 17 to hair.

Aspect 19 is the formulation of any one of Aspects 1-16, wherein the formulation includes less than 300 ppm fluoride as determined by ion-selective electrode following storage of the formulation in a sealed container at 50° C. for a period of three months.

Aspect 20 is a method of preparing the formulation of any one of Aspects 1-17, comprising contacting the polymer with the solvent, adding the amine neutralizer, and adding the propellant.

Aspect 21 is the formulation of any one of Aspects 1-10, wherein the fluoroolefin is present in the formulation in an amount of 10 wt.% to 90 wt.% based on the total weight of the formulation.

Aspect 22 is the formulation of any one of Aspects 1-11, wherein the solvent is present in an amount of 10 wt.% to 90 wt.% based on the total weight of the formulation.

What is claimed is:

1. A formulation comprising:
a fluoroolefin of the formula 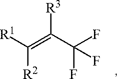
wherein R¹ is hydrogen or fluorine;
R² is hydrogen or fluorine;
R³ is hydrogen or fluorine;
a solvent;
an amine neutralizer; and
a fixative polymer.

2. The formulation of claim 1, wherein the fluoroolefin has a GWP, as measured in carbon dioxide (CO₂) equivalents, of 10 or less.

3. The formulation of claim 1, wherein the fluoroolefin comprises trans-1,3,3,3-tetrafluoropropene (HFO-1234ze(E)).

4. The formulation of claim 1, wherein the solvent comprises ethanol.

5. The formulation of claim 1, wherein the amine neutralizer comprises an amine that is unreactive towards trans-1,3,3,3-tetrafluoropropene (HFO-1234ze(E)) as determined by detection of fluoride via an ion-selective electrode.

6. The formulation of claim 1, wherein the amine neutralizer comprises a tertiary amine.

7. The formulation of claim 1, wherein the amine neutralizer comprises triethanolamine (TEA), triisopropanolamine (TIPA), or a mixture thereof.

8. The formulation of claim 1, further comprising at least one of one or more co-propellants and one or more co-solvents.

9. The formulation of claim 1, further comprising one or more additives selected from the group consisting of lubricants, stabilizers, corrosion inhibitors, fragrances, starches, and performance modifiers.

10. The formulation of claim 1, wherein the fixative polymer is selected from the group consisting of acrylate-octylacrylamide copolymers, vinyl acetate-crotonate-vinyl neodecanoate co-polymers, and octylacrylamide-acrylate-butylaminoethyl-methacrylate copolymers.

11. The formulation of claim 1, wherein the fluoroolefin is present in the formulation in an amount of 10 wt.% to 90 wt.% based on the total weight of the formulation.

12. The formulation of claim 1, wherein the solvent is present in an amount of 10 wt.% to 90 wt.% based on the total weight of the formulation.

13. The formulation of claim 1, wherein the amine neutralizer is present in the formulation in an amount of 0.2 wt.% to 1.5 wt.% based on the total weight of the formulation.

14. The formulation of claim 1, wherein the fixative polymer is present in the formulation in an amount of 1 wt.% to 15 wt.% based on the total weight of the formulation.

15. A personal care formulation comprising the formulation of claim 1.

16. The personal care formulation of claim 15, wherein the formulation is an aerosol.

17. The personal care formulation of claim 15, wherein the formulation is a hairspray.

18. A method, comprising applying the formulation of claim 17 to hair.

19. The formulation of claim 1, wherein the formulation includes less than 300 ppm fluoride as determined by ion-selective electrode following storage of the formulation in a sealed container at 50° C. for a period of three months.

20. A method of preparing the formulation of claim 1, comprising:
contacting the polymer with the solvent;
adding the amine neutralizer; and
adding the propellant.
